(19) United States
(12) Plant Patent Application Publication
(10) Pub. No.: US 2008/0134378 P1
(43) Pub. Date: Jun. 5, 2008

(54) 'VIBEHU99-1' plum tree

(76) Inventors: W. Vincent Hutcheson, Visalia, CA (US); Betty J. Hutcheson, Visalia, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 11/541,104

(22) Filed: Sep. 29, 2006

(51) Int. Cl. A01H 5/00

(52) U.S. Cl. PLT/184

(57) ABSTRACT

A new and distinct variety of plum tree which is distinguished by producing fruit that is mature for harvesting and shipment approximately June 1 to June 12 in the southern San Joaquin Valley of central Calif., more specifically in Tulare County, Calif., and wherein the fruit is a very dark red color, almost black, with yellow flesh. The fruit does not bruise easily and has very good flavor. The Fruit is able to retain its firmness and good flavor even after thirty days in cold storage.

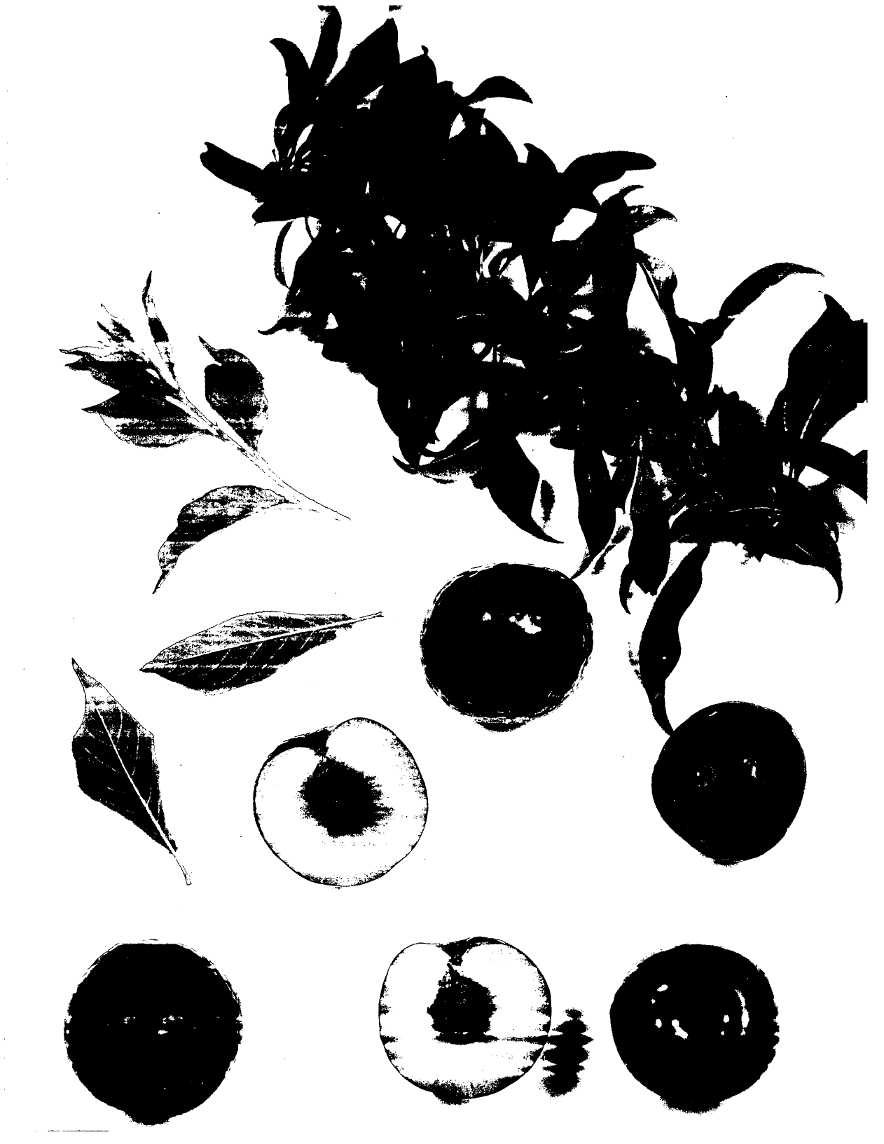

### CROSS-REFERENCE TO RELATED APPLICATIONS

None.

### BACKGROUND OF THE NEW VARIETY

1. Field of the Invention

The present invention relates to a new and distinct variety of plum tree, which will hereinafter be denominated varietally as the ‘VIBEHU99-1’ plum tree. More particularly, the present invention relates to such a plum tree that produces an early plum with a harvest date near the first of June in the southern San Joaquin Valley of central Calif. with good flavor and shipping quality.

2. Background

In the development of new commercial varieties of fruit trees, many growers, processors and sellers place a premium on early or late maturing of the fruit produced by the tree. However, some of the known earlier or later varieties produce fruit which are of small size, lack good flavor or do not have the desired coloration, characteristics which decrease the commercial success of the variety. A new and marketable variety of fruit must have the desired size, flavor and color characteristics and, preferably, have a different maturity date than that of similar fruit to expand the market for the fruit. The present invention provides a plum tree that meets all of the aforementioned criteria and, therefore, is believed to be of commercial appeal to those in the industry.

### SUMMARY OF THE NEW VARIETY

The ‘VIBEHU99-1’ plum tree is characterized by producing an early harvest plum that is dark in color with yellow flesh which is ripe for commercial harvesting and shipment in the first part of June in Visalia, Calif. (Tulare County, Calif., also referred to generally as the southern San Joaquin Valley). The ‘VIBEHU99-1’ plum tree resulted from a chance sport from an Angelino tree in an orchard in Visalia, Calif. The plum produced by the tree is approximately 2-⅜ inch diameter or larger, substantially round in shape and nearly black in color. The flesh of the fruit is almost clear yellow with very little red in the pit cavity. At harvest, the fruit from the new tree variety handles well and, due to the firm crisp nature of the plum, it will ship very well. The fruit has a very good flavor and has been determined to have outstanding storage life, retaining its firm flesh and good flavor even after thirty days in cold storage.

### ORIGIN AND ASEXUAL REPRODUCTION OF THE NEW VARIETY

The plum tree of the present invention was discovered by the inventors as a chance sport from an Angelino tree at their orchard in Visalia, Calif. in the San Joaquin Valley of central Calif. The first fruit was seen on this seedling in the Spring of 1999. The inventors noted that the fruit had the size, shape and color desired for a plum they were looking to develop. The fruit also had an early harvest time, a characteristic the inventors were also seeking. The fruit has a yellow flesh and is a cling seed. As the fruit matured, it was examined as to size, shape, coloration, ripening date, flavor, texture and as to other criteria which were sought in a plum variety having the desired commercial potential. Because the plum had the potential to be available early, having been seen on a seedling tree in early June, the seedling was marked for further testing and development.

Bud wood was collected from the sport and other seedling were budded. The harvest time for the fruit from the asexually produced trees of this new variety was approximately early June, about the same time as the sport tree. The budded trees also produced fruit of the same size, color and texture as that from the sport tree. As with the sport tree, the flavor of the fruit produced from the budded trees was very good. The skin coloration of the fruit was the same very dark red, almost black, and the flesh was the same almost clear yellow as the sport tree. In addition, the leaves, bark and growth pattern characteristics of the budded trees also matched the sport tree. The inventors have confirmed that the asexually reproduced plum trees of the new variety are the same in all respects to the sport tree.

### BRIEF DESCRIPTION OF THE DRAWING

The drawing is a color photograph showing mature fruit of the new variety including a first in side elevation, a second in bottom plan view showing the apex area thereof, a third in top plan view showing the base thereof, a fourth in side elevation showing the suture thereof and a fifth sectioned and laid open to show the stone in one section and the stone cavity in the other section. In addition, the color photograph includes representative foliage, all of the new variety.

### DETAILED DESCRIPTION

Referring more specifically to the pomological details of this new and distinct variety of plum tree, the following characteristics have been observed under the ecological conditions prevailing at the Hutcheson farm, the orchard of origin, which is located near Visalia, Calif. All major color code plate designations are by reference to the color name chart of the Inter-Society Color Council, National Bureau of Standards. Common color names are also occasionally employed.

### TREE

Generally:
   Size.—Normal plum tree size with up-right growth.
   Vigor.—Very vigorous.
   Chilling requirements.—Normal, approximately four (4) to six (6) hundred hours, in Tulare County of central Calif.
   Figure.—Normal or in any manner of pruning and shaping for good harvest.
   Productivity.—Very good.
   Regularity of bearing.—Will bear well every year, this variety seems to be self-fruiting.
Trunk:
   Size.—Normal diameter, depending on pruning and shaping.
   Surface texture.—Very rough bark.
   Color.—Yellow brown (73-P.OY).
   Lenticels on trunk.—Very rough with long vertical lenticels 6 inches (15.2 cm) to 12 inches (30.5 cm) long, color yellow brown (73-P.OY).
   Small lenticels between large lenticels.—Number — 8 to 10 per square inch.
   Small lenticels between large lenticels.—Size — ¼ inch (0.64 cm). Branches:
   Size.—Normal, depending on pruning and shaping.
   Surface texture.—Not as rough as trunk.
   Color.—Light brown (47 d.g.y.b.).
   Lenticels.—Size — Small, ¼ inch (0.64 cm) long and ⅛ inch (0.32 cm) wide.
   Immature branches.—Color — Bright green (117 s.y.g.).

### LEAVES

General information: This tree sets a very heavy crop of leaves, with 6 to 7 leaves on each spur on mature branches, 2 inches (5.08 cm) to 4 inches (10.2 cm) in length. Spurs are approximately 1 inch (2.54 cm) apart on mature branches.
Size:
   Average length.—2-¼ inches (5.72 cm) to 3-¼ inches (8.26 cm).
   Average width.—¾ inch (1.91 cm) to 1-½ inch (3.81 cm).
Form: Lanceolate.
Color:
   Upwardly disposed surface.—Dark green (126 d.01.g.).
   Downwardly disposed surface.—Light green (125 m.01.g).
Marginal form: Very finely serrate.
Glandular characteristics: None found on stem.
Petiule:
   Length.—½ inch (1.27 cm).
   Width.— 1/16 inch (0.160 cm).
Stem glands: None found.
Stipules: None found.

### FLOWER BUDS

General: Information was taken as white started to show at apex of the bud.
Size:
   Length.— 3/16 inch (0.476 cm).
   Diameter.— 3/16 inch (0.476 cm).
Shape: Almost round in appearance.
Texture: Smooth.
Petiole length: 3/16 inch (0.476).
Color: Bright green (117.s.y.g.), color at apex — white (263.white).
Other characteristics: Fruit spurs are close together with 2 to 6 buds per spur. Spurs are approximately 1 inch (2.54 cm).

### FLOWERS

Date of bloom: About February 22^{nd} to the 24^{th} at Visalia area in central Calif.
Size:
   Petal.—Length.— 3/16 inch (0.476 cm).
   Petal.—Diameter.— 3/16 inch (0.476 cm).
Shape: Each petal very small and round in appearance.
Petals color: White (263.white).
   Petiole.—Length — 3/16 inch (0.476 cm).

### FRUIT

General information: Maturity when described ripe for commercial harvesting and shipment about June 1^{st} to June 12^{th} in Tulare County of central Calif.
Size:
   Generally.—Medium.
   Average diameter.—Approximately 1-¾ inches (4.4 cm) to 2-⅜ inches (6.0 cm).
Form:
   Uniformity.—Good.
   Symmetrical.—Yes. Almost full round in appearance and rounded at the base and apex.
Suture:
   Generally.—Smooth, very slightly recessed.
   Length.—Approximately 2-¾ inches (6.9 cm) to 3-⅛ inches (7.65 cm) from stem cavity to apex area.
Ventral surface: Rounded.
Stem cavity:
   Generally.—Rounded.
   Width.— 5/16 inch (0.794 cm).
   Depth.—¼ inch (0.65 cm).
Pistil point: Apex rounded.
Stem:
   Generally.—Normal.
   Length.—½ inch (1.27 cm).
   Width.— 3/32 inch (0.238 cm).
Skin:
   Thickness.—Approximately 1/64 inch (0.040 cm) or less.
   Texture.—Very smooth.
   Tendency to crack.—None.
   Color.—Very dark red, almost black (260 v.d.pr) full color.
Flesh:
   Flesh color.—Mostly yellow, with light touch of green (104 p.9y).
   Surface of pit cavity.—Rough.
   Color of pit cavity.—Same as flesh.
   Juice production.—Medium juicy.
   Flavor.—Mild.
   Aroma.—Slight.
   Texture.—Crisp and juicy at harvest time.
   Fibers.—None.
   Ripening.—Even.
   Eating quality.—Good at harvest time, crisp and juicy.
Stone:
   Attachment.—Not full, free stone at harvest time.
   Fiber.—None.
   Size.—Length.— ¾ inch (1.91 cm).
   Size.—Width.— 1 1/16 inch (1.75 cm).
   Size.—Thickness.— ⅜ inch (0.95 cm).
   Form.—Elongated, almost flat in appearance.
   Apex.—Shape.— Rounded.
   Color.—Light Brown (74 s.y.b.).
   Sides.—Rough.
   Ridges.—None.
   Tendency to crack.—None.
Use: Fresh fruit market.
Keeping quality: Very good, has kept well in cold storage for 25 to 30 days and has not lost its flavor.
Resistence to disease: No disease noted to date.
Harvesting: Fruit is very firm at harvest date.
Shipping and handling qualities: Due to the firm nature of the fruit at harvest time, it will handle and ship very well.

Although this new variety of plum tree possesses the described characteristics noted above as a result of the growing conditions prevailing near and in the southern part of the San Joaquin Valley of Calif., more specifically in Tulare County, Calif., it is understood that variations of the usual magnitude and characteristics incidental to changes in growing conditions and horticultural practices, including but not limited to irrigation, fertilization, pruning, pest control, climatic variation and the like, are to be expected. It should also be realized by those skilled in the art that such variations do not depart from the spirit and scope of the invention as set forth in the appended claim.

What is claimed is:

1. A new and distinct variety of plum tree substantially as illustrated and described herein, with the characteristics of an early plum with a harvest date at about the first of June in the San Joaquin Valley of central Calif. with good flavor and shipping quality.
